(19) United States
(12) Patent Application Publication
(10) Pub. No.: US 2023/0285495 A1
(43) Pub. Date: Sep. 14, 2023

(54) METHODS, ANHYDROUS COMPOSITIONS AND APPLICATORS FOR DELIVERY OF NUTRITIONAL SUPPLEMENTS WHILE CONCURRENTLY/SIMULTANEOUSLY PROVIDING AROMATHERAPY

(71) Applicant: Boostceuticals, LLC, Wayne, PA (US)

(72) Inventors: Jay Tapper, Wayne, PA (US); Kristien Del Ferraro, Stamford, CT (US); Celeste Lutrario, Cary, NC (US); John Buchanan Replogle, Raleigh, NC (US)

(73) Assignee: Boostceuticals, LLC, Wayne, PA (US)

(21) Appl. No.: 17/690,506

(22) Filed: Mar. 9, 2022

(51) Int. Cl. A61K 36/898; A61K 9/00; A61K 36/28; A61K 36/534; A61K 36/537; A61K 36/53; A61K 36/752; A61K 36/35; A61K 36/258; A61K 36/81; A61K 36/185; A61K 36/31; A61M 21/02; A61M 35/00

(52) U.S. Cl. CPC A61K 36/898; A61K 9/0053; A61K 9/007; A61K 9/0014; A61K 36/28; A61K 36/534; A61K 36/537; A61K 36/53; A61K 36/752; A61K 36/35; A61K 36/258; A61K 36/81; A61K 36/185; A61K 36/31; A61M 21/02; A61M 35/003; A61M 2021/0016

(57) ABSTRACT

Methods, anhydrous compositions, and applicators, for delivery of nutritional supplements while concurrently providing aromatherapy. It finds particular application in conjunction with the oral/lip delivery and absorption and/or ingestion of natural botanical nutritional supplements into a human body, while concurrently providing natural botanical aromatherapy to a user as the natural botanical nutritional supplements are self-administrated by the user to the user's lips and will be described with particular reference thereto.

### BACKGROUND

The present exemplary embodiments relate to methods, anhydrous compositions, and applicators, for delivery of nutritional supplements while concurrently providing aromatherapy. It finds particular application in conjunction with the oral/lip delivery and absorption and/or ingestion of natural botanical nutritional supplements into a human body, while concurrently providing natural botanical aromatherapy to a user as the natural botanical nutritional supplements are self-administrated by the user to the user's lips and will be described with particular reference thereto. However, it is to be appreciated that the present exemplary embodiment is also amenable to other like applications, and other supplements and aromatherapy ingredients not necessarily including the ingredients described herein.

Lip balms, including natural and non-naturally derived ingredients are commonly used to treat chapped lips. Commonly, these lip balms include ingredients to hydrate the lip skin surface and are applied directly to the treated surface. U.S. Pat. No. 10,624,837 discloses a food-grade lip balm for use in treating drying and peeling lip skin.

Nutritional supplements are commonly taken by people to supplement their nutritional intake on a daily basis. These generally include vitamins, minerals, herbs, amino acids, and enzymes, and are taken by mouth. U.S. Pat. No. 6,517,818 discloses a lip balm that includes the delivery of vitamins (for example vitamin C) to the lip skin of the user for localized skin care. In other words, the vitamins contained within the balm and applied to the lip skin are selected to promote skin healing at the area of application.

Topical essential oil scentsare used, in part, for their aromatherapeutic benefits. Essential oils are the natural, aromatic volatile liquids found in shrubs, flowers, trees, resins, fruit peels, rhizomes, roots, bushes, and seeds. Essential oil scents can be extracted from aromatic plant sources via steam distillation, cold pressing, and other types of distillation. Essential oil scents are highly concentrated and far more potent than dry herbs. Other topically applied oils used include olive oil, almond oil, coconut oil, etc. and oils high in esters, such as jojoba oil, and waxes such as beeswax.

While essential oil scents often have a pleasant aroma, their chemical makeup is complex and their benefits vast which makes them much more than something that simply smells good. Historically, essential oils have played a prominent role in everyday life. Today, essential oils are used for aromatherapy, massage therapy, emotional health, and personal care. US Published Patent Application 2013/0064596 discloses a roller-ball applicator for applying essential oils to a user's skin.

Provided herein, and lacking in the prior art, is a novel and nonobvious balm composition for application to a user's lip skin which 1) provides for delivery of nutritional supplement absorption and/or ingestion into the user's body for a wellness benefit distinct from any topical benefit related to a physical condition of the user's lips, and simultaneously 2) provides nasal aromatherapy to the user with, for example, a botanical essential oil scent portion included in the balm or ointment composition.

In contrast, and distinguishable from common lip balm and essential oil delivery devices and compositions, the disclosed novel lip balm/ointment composition includes specific proportions, i.e. ratios, of an oil base portion amount, essential oil scent portion amount and nutritional supplement portion amount, relative to a total balm and/or ointment composition amount, where the oil base portion amounts to at least 50% of a total weight of the balm and/or ointment composition, the essential oil scent portion amounts to 30% or less of the total weight of the balm and/or ointment composition, and the natural botanical nutritional supplement portion amounts to 20% or less of the total weight of the balm and/or ointment composition. The disclosed balm and ointment composition and associated proportional properties, when applied to a user's lips, provides a user with nutritional supplement absorption and/or ingestion into the user's body for a wellness benefit distinct from any topical benefit related to a physical condition of the user's lips, such as that provided by traditional lip balms. In addition, the essential oil scent, applied to the user's lips during an application of the balm or ointment including the nutritional supplement, also provides nasal aromatherapy to the user simultaneously with nutritional supplement absorption and/or ingestion into the user's body.

In addition to providing simultaneous delivery of nutritional supplements and aromatherapy, the disclosed balm/ointment composition proportional formulations can be applied freely by a user throughout the day, for example but not limited to 3-7 times day, without any risks of absorbing and/or ingestion more than a daily recommended dose of the nutritional supplement.

To summarize, distinguishable from conventional lip balms and conventional essential oil delivery devices and associated compositions, which ONLY either deliver a moisturizing lip balm OR essential oil, this disclosure, and the exemplary embodiments provided herein, provide a single product that provides concurrent/simultaneous application and delivery of a nutritional supplement(s) AND aromatherapy AND a moisturizing lip balm.

### BRIEF DESCRIPTION

According to an exemplary embodiment of this disclosure, disclosed is a method for a user to receive a self-administered oral dose of a natural botanical nutritional supplement while simultaneously receiving nasal aromatherapy, the method including: the user applying one of a balm and an ointment of anhydrous composition to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition, wherein, after applying the anhydrous composition the user's lips, at least a part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

According to another exemplary embodiment of this disclosure, disclosed is an anhydrous composition applied to a user's lips to orally deliver a nutritional supplement and simultaneously nasally deliver aromatherapy, the anhydrous composition including: one of a balm and an ointment of anhydrous composition applied to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition, wherein, after applying the anhydrous composition the user's lips, at least part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

According to another exemplary embodiment of this disclosure, disclosed is a pocket-sized therapeutic applicator to orally deliver botanical nutritional supplements to a user while simultaneously providing aromatherapy to the user, the applicator including: one of a balm and an ointment of anhydrous composition applied to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition; and an applicator housing containing the anhydrous composition and dispensing the anhydrous composition for application to the user's lips, wherein, after applying the anhydrous composition to the user's lips, at least part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

### BRIEF DESCRIPTION OF THE DRAWINGS

FIG. 1 is a flow chart of a method for oral/lip delivery of anhydrous compositions for absorption and/or ingestion of natural botanical nutritional supplements into a human body, while concurrently providing natural botanical aromatherapy according to exemplary embodiments of this disclosure;

FIG. 2 is a flow chart of a method/process for generating anhydrous compositions for oral/lip delivery and absorption and/or ingestion of natural botanical nutritional supplements into a human body, while concurrently providing natural botanical aromatherapy according to exemplary embodiments of this disclosure;

FIGS. 3A-3E show a balm applicator for providing anhydrous compositions for oral/lip delivery and absorption and/or ingestion of natural botanical nutritional supplements into a human body, while concurrently providing natural botanical aromatherapy according to exemplary embodiments of this disclosure.

### DETAILED DESCRIPTION

Disclosed is a method for users to self-administer nutritional supplements via the lips and mouth while simultaneously receiving aromatherapy via the nasal passageway via a balm or ointment composition specifically applied to the lips. More specifically, also disclosed is an anhydrous composition applied to the lips, using an applicator, for oral delivery of nutritional supplements concurrently with nasal delivery of aromatherapy scents.

According to an exemplary embodiment, the disclosed methods, anhydrous compositions and applicators include 1) delivering no more than 25% of the daily recommended dose of nutritional supplement per application, and 2) the anhydrous compositions include a natural wax and oil base, natural botanical essential oil scents, and natural botanical nutritional supplements, wherein the wax and oil base a) make up the majority (above 50%) of the total composition, b) the natural botanical essential oil scents make up the second largest portion (up to 30%) of the formula, c) the natural botanical nutritional supplements make up the smallest portion (up to 20%) of the formula, and d) the natural botanical essential oil scents in the formula are at least 5×, and no more than 15×, the natural botanical nutritional supplement ingredients in relation to the overall formula.

The anhydrous composition is dispensed in a portable, pocket size applicator to be applied to the lips as needed/desired, for example, but not limited to, a stick container with wheel and threaded rod with base mechanism and cap, a squeezable tube container with pinhole dispenser mechanism and cap, or a jar container including a removable top.

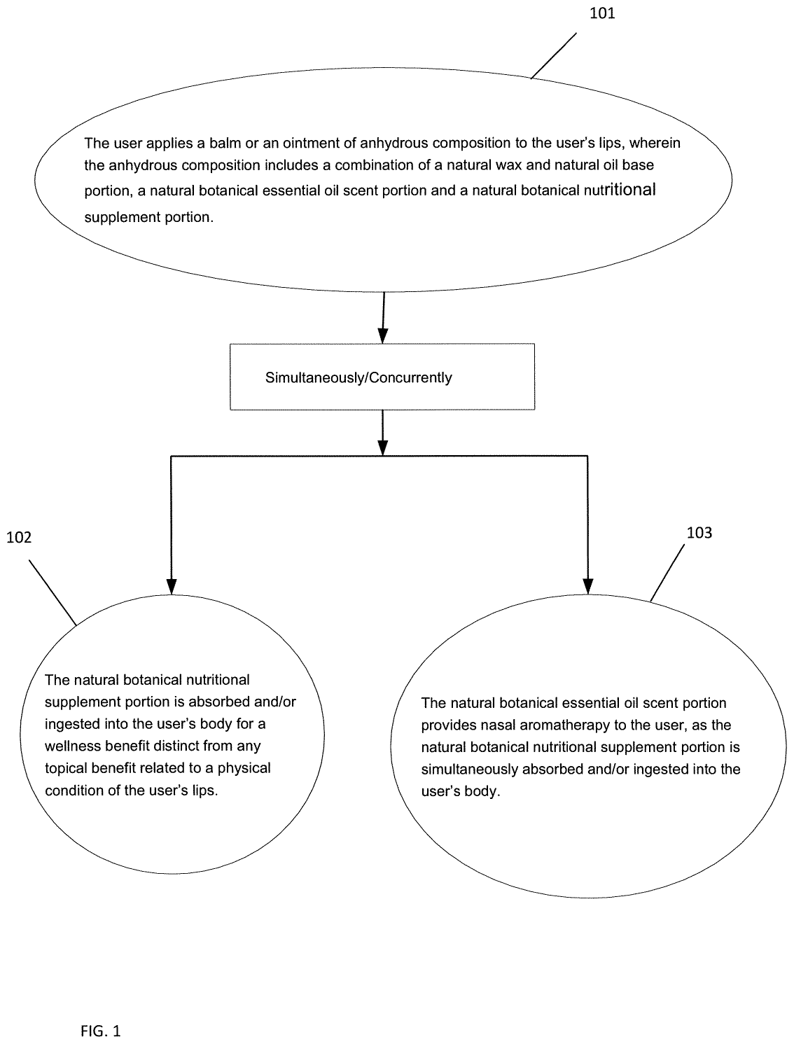

With reference to FIG. 1, shown is a flow chart of a method for oral/lip delivery of anhydrous compositions for absorption and/or ingestion of natural botanical nutritional supplements into a human body, while concurrently providing natural botanical aromatherapy.

Initially, at step 101, the user applies or self-administers a balm or an ointment of anhydrous composition to the user's lips. The anhydrous composition includes a combination of a natural wax and natural oil base portion, a natural botanical essential oil scent portion and a natural botanical nutritional supplement portion. As will be further described below, the natural wax and natural oil base portion amounts to at least 50% of a total weight of the anhydrous composition, the natural botanical essential oil scent portion amounts to 30% or less of the total weight of the anhydrous composition, and the natural botanical nutritional supplement portion amounts to 20% or less of the total weight of the anhydrous composition.

After the anhydrous composition is applied to the user's lips, simultaneously/concurrently, at step 102, the natural botanical nutritional supplement portion is absorbed and/or ingested into the user's body for a wellness benefit distinct from any topical benefit related to a physical condition of the user's lips, AND, at step 103 the natural botanical essential oil scent portion simultaneously provides nasal aromatherapy to the user.

The anhydrous composition applied to the user's lips is now described with particular reference to Wellness Benefits associated with Sleep, Move (Increasing Energy), Calm (Relieving Stress), Smile (Mood Improvement), and Love (Increasing Libido). However, it is to be understood that other wellness benefits, including but not limited to, Immunity, Craving control, Beauty, Focus/Brain are within the scope of this disclosure and the methods, compositions and applicators embodiments disclosed herein.

To provide simultaneous delivery of nutritional supplements and aromatherapy, specific proportions of balm base/essential oil scent/nutritional supplement content, relative to the total mass or weight of the balm, are adhered to provide a desired wellness benefit. The wellness benefit is achieved with the simultaneous delivery of the nutritional supplements to the body of the user and nasal delivery of aromatherapy via the oil/scent contained within the balm and applied to the user's lips. The anhydrous composition includes a combination of a 1) natural wax and natural oil base portion, a 2) natural botanical essential oil scent portion and a 3) natural botanical nutritional supplement portion. To orally deliver a nutritional supplement and simultaneously nasally deliver aromatherapy to a user, the natural wax and natural oil base portion amounts to at least 50% of a total weight of the anhydrous composition, the natural botanical essential oil scent portion amounts to 30% or less of the total weight of the anhydrous composition, and the natural botanical nutritional supplement portion amounts to 20% or less of the total weight of the anhydrous composition.

### Natural Wax and Natural Oil Base Portion

The natural wax and natural oil base portion includes, but is not limited to:
Oils Helianthus Annuus (Sunflower) Seed Oil, Cocos Nucifera (Coconut) Oil, Ricinus Communis (Castor) Seed Oil and Meadowfoam Seed Oil; waxes Beeswax, Euphorbia Cerifera (Candelilla) Wax and Carnauba Wax; butters Butyrospermum Parkii (Shea) Butter, Theobroma Cacao (Cocoa) Seed Butter, and Kokum Butter, and other ingredients Squalene, Mica, Tocopherol and Hydrogenated Coco-Glycerides.

Alternative botanical oils may include, but are not limited to, canola oil, soybean oil, petroleum, argan oil, jojoba oil, avocado oil, olive oil, maracuja, and almond oil.

Alternative botanical waxes may include, but are not limited to, lanolin, paraffin, ozokerite, and jojoba wax.

Alternative botanical butters may include, but are not limited to, mango butter and avocado butter.

Alternative other ingredients may include, but are not limited to, ethylhexyl palmitate, trimethicone, dimethicone and aloe.

### Natural Botanical Essential Oil Scent Portion and Natural Botanical Nutritional Supplement Portion

Table 1 below lists examples of ingredients included in, at least in part, the natural botanical essential oil scent portion, and the natural botanical nutritional supplement portion. As indicated in the table, the ingredients included are associated with the wellness benefits of Sleep, Move, Love, Calm and Smile.

TABLE 1

| | SLEEP; | MOVE; | LOVE; | CALM; | SMILE; |
|---|---|---|---|---|---|
| | Soothing | Ener- | Tanta- | Relieving | Uplifting |
| | blend | gizing | lizing | blend | blend to |
| | to ease into | blend | blend | to calm | elevate |
| Wellness | restful | to get you | to set | your | your |
| Benefit: | sleep | moving | the mood | mind | mood |
| Botanical | Chamomile | Zesty | Vanilla | Spearmint | Lime |
| Therapeutic | Lavender | Grapefruit | Cocoa | Pepper- | Clary |
| Scents and | | Orange/ | | mint | Sage |
| Flavors: | | Mandarin | | | |
| Botanical | Valerian | Ginseng | Maca | Ash- | St. John's |
| Nutritional | Root | | Root | wagandha | Wart |
| Supplement: | | | | | |

Alternative natural botanical scents and flavors may include, but are not limited to the following:
Sleep: Bergamot, Clary Sage, Marjoram, Frankincense, Jasmine, Ylang, Ylang, Sandalwood, Peppermint, Marjoram, Cedarwood, Clary Sage, Vetiver, Valerian, Eucalyptus and Neroli.
Move: Lemon, Peppermint, Cedarwood, Balsam Fir, Rosemary, Cinnamon, Basil, Tea Tree, Tangerine, Lime, and Bergamot.
Calm: Lemon, Lavender, Lemongrass, Ylang-Ylang, Bergamot, Neroli, Yuzu, Jatamansi, Valerian, Jasmine, Basil, Rose, Vetiver, Patchouli, Geranium, and Frankincense.
Smile: Bergamot, Frankincense, Vetiver, Lavender, Sage, Cedarwood, Sandalwood, Jasmine, Rose, Ylang-Ylang, Black Pepper, Orange, Peppermint, Spearmint, Basil, and Lemon.
Love: Cinnamon, Ylang-Ylang, Rose, Palmarosa, Patchouli, Jasmine, Rose, Sandalwood, Clary Sage, Ginger, Neroli, Nutmeg, Hong Kuai, and Geranium. Immunity: Lavender, Lemon, Oregano, Tea Tree, Eucalyptus, Ginger, Thyme, and Rosemary.
Cravings: Cinnamon, Anise, Peppermint, Basil, Orange, Grapefruit, Juniper, Fennel, Lavender, Ginger, Sage, Lemongrass, and Bergamot.

### Beauty: Rose, Coconut, Vanilla, Tea Tree, Bergamot, Cypress, Orange, Rosemary, Jasmine, and Lavender.

Focus/Brain: Peppermint, Spearmint, Rosemary, Lemon, Orange, Vetiver, Lavender, Cedarwood, Basil, Lemongrass, Frankincense, Cypress, Patchouli, Juniper, Pine, and Clarity.

Alternative Synthetic scents (not natural botanical) may also be used according to other embodiments of this disclosure. Specific wellness benefit formulas can use much less synthetic scent, relative to botanical natural scents, to get an equivalent strength of smell/fragrance relative to botanical scents. Non-botanical supplements (like vitamins, minerals, amino acids, enzymes, etc.), include, but are not limited to:
Sleep: L-Theanine, 5-HTP, Melatonin, Gaba, and Magnesium.
Move (Energy): Creatine, B12, and CoQ10.
Calm: 5-HTP, Gaba, L-Theanine, Magnesium, and Vitamin D.
Smile (Mood): Gaba, 5-HTP, Inositol, SAMe, Omega 3 and Vitamin D.
Love : DHEA, L-Citrulline, and L-Arginine.
Immunity (Boosting): Vitamin E, Vitamin C, Vitamin D, Zinc, and Quercetin.
Cravings (Suppression): L-tryptophan, L-carnosine, Co Q10, Lipase, L-Glutamine, Chromium, and Vitamin B12.
Beauty: Hyaluronic Acid, Keratin, Biotin, Collagen, Folic Acid, Vitamin B12, Vitamin D, and Vitamin C.
Focus/Brain: Acetyl-L-Carnitine, Creatine, Phosphatidylserine, Fish Oils, Omega 3, Alpha GPC, Vitamin B Complex, and Resveratrol.

Alternative natural botanical nutritional supplements may include, but are not limited to the following:
Sleep: Tart Cherry Extract, CBD, Passionflower, Poppy, Lemon Balm, Jujube, Chamomile, Lavender, Kava Root and Ashwagandha.
Move: Beetroot, Coffee bean/berry, Teas (all incl. matcha), Yerba mate, Kola Nut, Goji Berry, and Rhodiola.
Calm: Rhodiola, CBD, Eucalyptus, Lemon Balm, Holy Basil, Passionflower, St. John's Wort, Lavender, and Chamomile.
Smile: Ginkgo Bilboa, Curcumin, Saffron, Magnolia, Passionflower, Ashwagandha, Damiana, and Lemon Balm.
Love: Pine Bark, Ginseng, Damiana, Ashwagandha, Epimedium/Horny Goat Weed, Avena Sativa, and Yohimbe.
Immunity: Elderberry, Echinacea, Beta-carotene, Green Tea, Baicalin, Ginger, and Curcumin.
Cravings: Goji Berry, Ginger, Green Tea, and Apple Cider Vinegar.
Beauty: Ginseng, Aronia Berry, Dandelion Root, Lutein, and Zeaxanthin.
Focus/Brain: Ginseng, Ginkgo Biloba, Caffeine, Bacopa Monnieri, and Rhodiola Rosea.

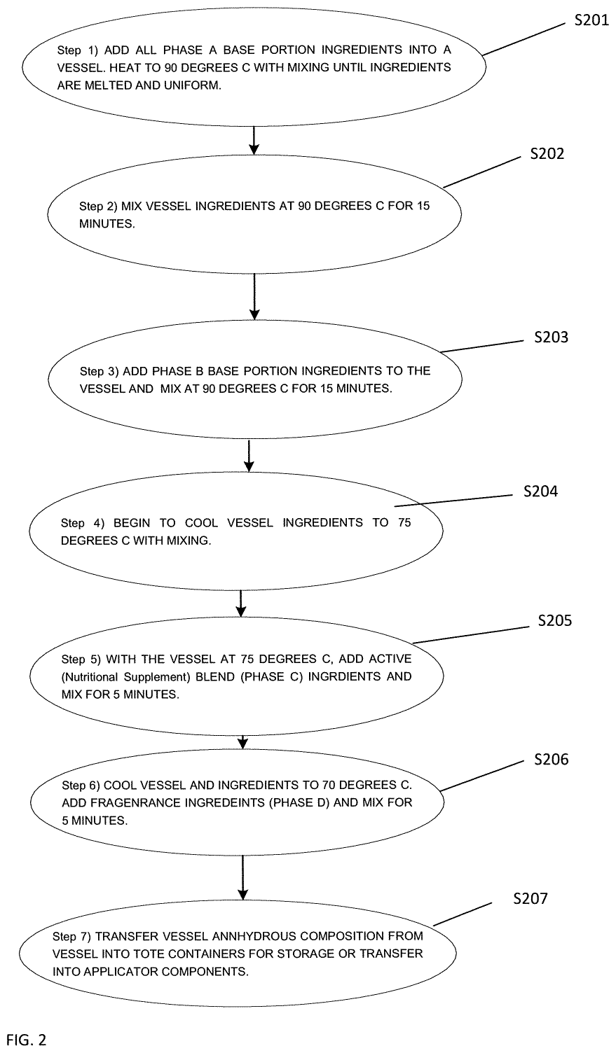

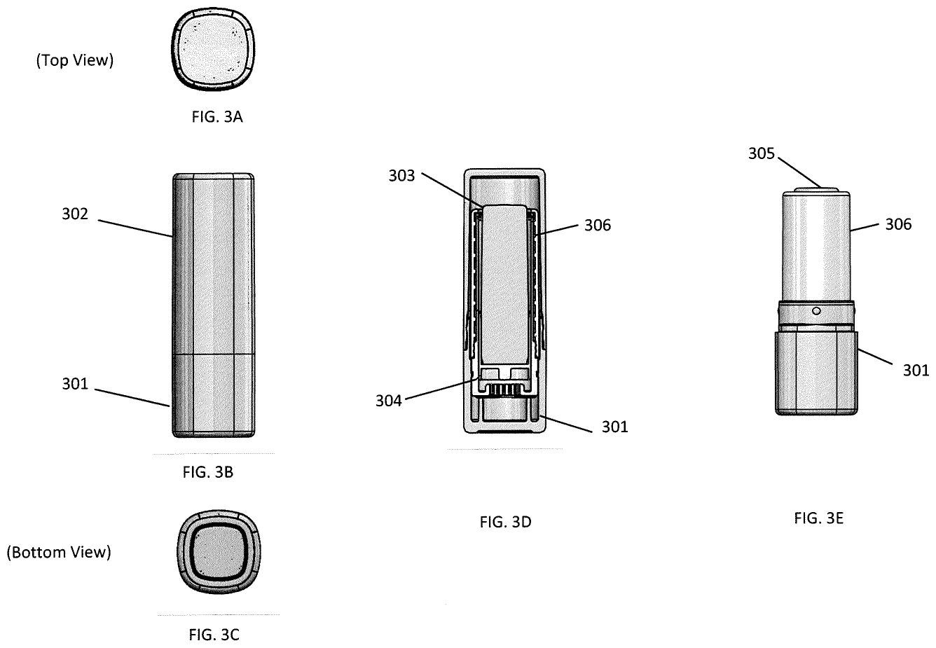

According to an exemplary embodiment, which will now be described with reference to FIGS. 2 and 3, the disclosed anhydrous compositions and associated applicator deliver no more than 25% of the daily recommended dose of nutritional supplement per application, (for example, but not limited to, the amounts listed in Table 2 below), using a lip balm applicator, such as but not limited to, a stick container actuated with a thumb wheel and threaded rod operatively associated with a base mechanism with a cap; a squeezable tube container operatively associated with a pinhole dispenser mechanism with a cap; a vial container operatively associated with a roller ball dispenser mechanism with a cap; or a jar container including a removable top.

TABLE 2
BOTANICAL NUTRITIONAL SUPPLEMENT

| BOOSTED | BOTANICAL NUTRITIONAL | MAX AVG. |
|---|---|---|
| SKU | SUPPLEMENT | DAILY DOSE |
| SLEEP | Valerian Root | Up to 600 mg daily |
| MOVE | Ginseng | Up to 3,000 mg daily |
| LOVE | Maca | Up to 3,500 mg daily |
| CALM | Ashwagandha | Up to 1,000 mg daily |
| SMILE | St. John's Wort | Up to 2,700 mg daily |

The anhydrous composition is made of a natural wax and oil base, a natural botanical essential oil scent portion, and a natural botanical nutritional supplement portion, including the ingredients as previously described. To provide the delivery of the nutritional supplements to a user's body, via the user's lips, while simultaneously providing nasal aromatherapy to the user, the proportions of the base portion, scent portion and supplements portion, relative to the total weight of the balm or ointment, is as follows:
a) the wax and oil base make up the majority (above 50%) of the total composition;
b) the natural botanical essential oil scents make up the second largest portion (up to 30%) of the formula;
c) the natural botanical nutritional supplements make up the smallest portion (up to 20%) of the formula; and
d) the natural botanical essential oil scents in the formula are at least 5×, and no more than 15×, the natural botanical nutritional supplement ingredients in relation to the overall formula.

For example, for a 4250 milligram (4.25 gram) lip balm stick, each lip balm stick contains 68 milligrams (0.068 grams) of a botanical supplement, and 680 milligrams (0.68 grams) of botanical essential oil scent for this example. (The essential oil scent portion is 10× the nutritional supplement portion) Therefore, 1.6% of the total lip balm composition includes the botanical nutritional supplement, i.e., 68 milligrams of the botanical nutritional supplement is included in each lip balm stick according to this example. In addition, 80% of a botanical nutritional supplement blend is a carrier oil base (e.g., sunflower oil) and 20% of the botanical nutritional supplement blend is botanical nutritional supplement. Therefore, the total amount of the botanical nutritional supplement blend amounts to 340 milligrams, including 272 milligrams of carrier oil and 68 milligrams of nutritional supplement. This botanical nutritional supplement blend (Phase C Active Blend below) is used during processing, as will be described below with reference to FIG. 2.

In other words, as claimed herein,
the natural wax and natural oil base portion amounts to at least 50% of a total weight of the anhydrous composition (4250 milligrams−340 milligrams of Active Blend−680 milligrams of botanical essential oil scent=3230 milligrams (3.23 grams) or 76% of the natural wax and natural oil base portion);
the natural botanical essential oil scent portion amounts to 30% or less of the total weight of the anhydrous composition (680 milligrams (0.068 grams) or 16% of the total weight of the anhydrous composition); and
the natural botanical nutritional supplement portion amounts to 20% or less of the total weight of the anhydrous composition (68 milligrams (0.068 grams) or 1.6% of the total weight of the anhydrous composition).

In addition to providing simultaneous delivery of nutritional supplements and aromatherapy, the disclosed balm/ointment composition proportional formulations can be applied freely by a user throughout the day, for example but not limited to 3-7 times day or more, without any risks of absorbing and/or ingestion more than a daily recommended dose of the nutritional supplement.

It is to be understood that this is merely one example of a lip balm stick according to this disclosure and other variants, such as a balm or ointment composition with a total greater or lesser mass, and the amount of essential oil scent is 5×-15× the amount of nutritional supplement, where the natural botanical essential oil scent portion amounts to 30% or less of the total weight of the anhydrous composition and the natural botanical nutritional supplement portion amounts to 20% or less of the total weight of the anhydrous composition.

With reference to FIG. 2, shown is a flow chart of a method/process for generating anhydrous compositions for oral/lip delivery and absorption/ingestion of natural botanical nutritional supplements into a human body, while concurrently providing natural botanical aromatherapy according to an exemplary embodiment of this disclosure.

For purposes of describing an exemplary process of generating an anhydrous composition as described herein, Phase A, Phase B, Phase C and Phase D, as listed below, are mixed/heated/cooled, etc. as further described below. However, it is to be understood other Phase A, Phase B, Phase C, and Phase D ingredients may be used as previously described.

Phase A Base Ingredients: Oils Helianthus Annuus (Sunflower) Seed Oil, Cocos Nucifera (Coconut) Oil, Ricinus Communis (Castor) Seed Oil and Meadowfoam Seed Oil; waxes Beeswax, Euphorbia Cerifera (Candelilla) Wax and Carnauba Wax; butters Butyrospermum Parkii (Shea) Butter, Theobroma Cacao (Cocoa) Seed Butter, and Kokum Butter, and other ingredients Squalene, and Hydrogenated Coco-Glycerides.

Phase B Base Ingredient: Mica, and Tocopherol.

Phase C Active Blend: Depends on the desired Wellness Benefit as described above. The Active Blend is a blend of a carrier oil and botanical nutritional supplement, which includes a blend of a carrier oil and a botanical nutritional supplement ingredient, for example, as indicated in Table 2. According to the example provided, Sunflower Oil is used as the carrier oil, however other alternative carrier oils may include, but are not limited to, Olive Oil, Argan Oil, Grape seed Oil, Coconut Oil, Castor Oil, Sweet Almond Oil, Apricot Kernel Oil, Jojoba Oil, Meadowfoam Seed Oil, Avocado Oil.

Phase D Fragrance: Depends on the desired Wellness Benefit as described above.

### The PROCESS (FIG. 2)

At Step 1) ADD ALL BALM/OINTMENT PHASE A BASE INGREDIENTS INTO A VESSEL. HEAT TO 90 DEGREES C WITH MIXING UNTIL ALL PHASE A BASE INGREDIENTS ARE MELTED AND UNIFORM (S201);
At Step 2) MIX PHASE A BASE AT 90 DEGREES C FOR 15 MINUTES (S202);
At Step 3) ADD PHASE B BASE PORTION TO THE VESSEL. MIX AT 90 DEGREES C FOR 15 MINUTES (S203);
At Step 4) BEGIN TO COOL VESSEL TO 75 DEGREES C WITH MIXING (S204);
At Step 5) WHEN THE PHASE A AND PHASE B BASE PORTION IS AT 75 DEGREES C, ADD PHASE C INGREDIENTS AND MIX FOR 5 MINUTES (S205);
At Step 6) COOL PHASE A, B, AND C MIXED INGREDIENTS TO 70 DEGREES C. ADD PHASE D INGREDIENTS. MIX FOR 5 MINUTES (S206); and
At Step 7) TRANSFER VESSEL ANNHYDROUS COMPOSITION FROM VESSEL INTO TOTE CONTAINERS FOR STORAGE OR TRANSFER INTO APPLICATOR COMPONENTS (S207).

With reference to FIGS. 3A-3E, shown is a balm applicator for providing anhydrous compositions for oral/lip delivery and absorption/ingestion of natural botanical nutritional supplements into a human body, while concurrently providing natural botanical aromatherapy according to exemplary embodiments of this disclosure.

The lip balm applicator includes an applicator base 301, cap 302, a balm stick 303, a thumb wheel and threaded rod arrangement 304 operatively associated with the base 301 to provide a rotating actuating movement of the balm stick upwardly to expose balm surface 305 for application to a user's lips. In addition, the thumb wheel and threaded rod arrangement 304 provide a rotating actuating movement of the balm stick downwardly i.e., into the applicator housing, to protect the balm surface 305 within the applicator and provide clearance for attachment of the cap 302. A balm casing 306 within the applicator remains fixed and slidably engages the balm stick 303 and protective cap 302.

Alternative applicator components/arrangements may include, but are not limited to, a squeezable tube container operatively associated with a pinhole dispenser mechanism with a cap; a vial container operatively associated with a roller ball dispenser mechanism with a cap; and a jar container including a removable top.

Disclosed herein are exemplary embodiments including, but not limited to the following:

[A1] A method for a user to receive a self-administered oral dose of a natural botanical nutritional supplement while simultaneously receiving nasal aromatherapy, the method comprising: the user applying one of a balm and an ointment of anhydrous composition to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition, wherein, after applying the anhydrous composition the user's lips, at least a part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

[A2] The method of paragraph [A1], wherein the natural botanical essential oil scent portion is a least 5× and no more than 15× of the natural botanical nutritional supplement portion.

[A3] The method of paragraph [A1], wherein a single application of the anhydrous composition applied to the user's lips amounts to 25% or less than a daily recommended dose limit of the natural botanical nutritional supplement.

[A4] The method of paragraph [A1], wherein at least 5% of the natural botanical nutritional supplement included in a single application of the anhydrous composition applied to the user's lips is absorbed and/or ingested into the user's body.

[A5] The method of paragraph [A1], wherein the natural botanical essential oil scent portion includes at least one of: Chamomile, Lavender, Zesty Grapefruit, Mandarin, Cacao, Vanilla, Lemon, Peppermint, Lime and Clary Sage.

[A6] The method of paragraph [A1], wherein the natural botanical nutritional supplement portion includes at least one of: Valerian Root, Ginseng, Maca, Ashwagandha and St. John's Wort.

[A7] The method of paragraph [A1], wherein the natural botanical essential oil scent portion and the natural botanical nutritional supplement portion include one of the combinations of: Chamomile, Lavender, and Valerian Root; Zesty Grapefruit, Mandarin and Ginseng; Cacao, Vanilla and Maca; Lemon, Peppermint and Ashwagandha; and Lime Clary Sage and St. John's Wort.

[A8] The method of paragraph [A1], wherein the applicator is one of the following: a stick container actuated with a thumb wheel and threaded rod operatively associated with a base mechanism with a cap; a squeezable tube container operatively associated with a pinhole dispenser mechanism with a cap; a vial container operatively associated with a roller ball dispenser mechanism with a cap; and a jar container including a removable top.

[A9] The method of paragraph [A1], wherein the wellness benefit includes at least one of: sleep, relaxation, stress-relief, anxiety relief, calm, energy, awakening, libido, sexual pleasure, and mood.

[A10] The method of paragraph [A1], wherein the natural botanical essential oil scent portion is a least 5× and no more than 15× of the natural botanical nutritional supplement portion; the natural botanical nutritional supplement included in a single application of the anhydrous composition to the user's lips amounts to 25% or less than a daily recommended dose limit; at least 5% of the natural botanical nutritional supplement included in a single application of the anhydrous composition to the user's lips is absorbed and/or ingested into the user's body, and a total natural botanical nutritional supplement content of the anhydrous composition included in an applicator is less than 100% of the daily recommended dose limit.

[B1] An anhydrous composition applied to a user's lips to orally deliver a nutritional supplement and simultaneously nasally deliver aromatherapy, the anhydrous composition comprising: one of a balm and an ointment of anhydrous composition applied to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition, wherein, after applying the anhydrous composition the user's lips, at least part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

[B2] The anhydrous composition according to paragraph [B1], wherein the natural botanical essential oil scent portion is a least 5× and no more than 15× the natural botanical nutritional supplement portion.

[B3] The anhydrous composition according to paragraph [B1], wherein a single application of the anhydrous composition applied to the user's lips amounts to 25% or less than a daily recommended dose limit of the natural botanical nutritional supplement.

[B4] The anhydrous composition according to paragraph [B1], wherein at least 5% of the natural botanical nutritional supplement included in a single application of the anhydrous composition applied to the user's lips is absorbed and/or ingested into the user's body.

[B5] The anhydrous composition according to paragraph [B1], wherein the natural botanical essential oil scent portion includes at least one of: Chamomile, Lavender, Zesty Grapefruit, Mandarin, Cacao, Vanilla, Lemon, Peppermint, Lime and Clary Sage.

[B6] The anhydrous composition according to paragraph [B1], wherein the natural botanical nutritional supplement portion includes at least one of: Valerian Root, Ginseng, Maca, Ashwagandha and St. John's Wort.

[B7] The anhydrous composition according to paragraph [B1], wherein the natural botanical essential oil scent portion and the natural botanical nutritional supplement portion include one of the combinations of: Chamomile, Lavender, and Valerian Root; Zesty Grapefruit, Mandarin, and Ginseng; Cacao, Vanilla and Maca; Lemon, Peppermint and Ashwagandha; and Lime Clary Sage and St. John's Wort.

[B8] The anhydrous composition according to paragraph [B1], wherein the anhydrous composition is applied to the user's lips using one of the following: a stick container actuated with a thumb wheel and threaded rod operatively associated with a base mechanism with a cap; a squeezable tube container operatively associated with a pinhole dispenser mechanism with a cap; a vial container operatively associated with a roller ball dispenser mechanism with a cap; and a jar container including a removable top.

[B9] The anhydrous composition according to paragraph [B1], wherein the wellness benefit includes at least one of: sleep, relaxation, stress-relief, anxiety relief, calm, energy, awakening, libido, sexual pleasure, and mood.

[B10] The anhydrous composition according to paragraph [B1], wherein the natural botanical essential oil scent portion is a least 5× and no more than 15× of the natural botanical nutritional supplement portion; the natural botanical nutritional supplement included in a single application of the anhydrous composition to the user's lips amounts to 25% or less than a daily recommended dose limit; at least 5% of the natural botanical nutritional supplement included in a single application of the anhydrous composition to the user's lips is absorbed and/or ingested into the user's body, and a total natural botanical nutritional supplement content of the anhydrous composition included in an applicator is less than 100% of the daily recommended dose limit.

[C1] A pocket-sized therapeutic applicator to orally deliver botanical nutritional supplements to a user while simultaneously providing aromatherapy to the user, the applicator comprising: one of a balm and an ointment of anhydrous composition applied to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition; and an applicator housing containing the anhydrous composition and dispensing the anhydrous composition for application to the user's lips, wherein, after applying the anhydrous composition to the user's lips, at least part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

[C2] The pocket-sized therapeutic applicator according to paragraph [C1], wherein the natural botanical essential oil scent portion is a least 5× and no more than 15× of the natural botanical nutritional supplement portion.

[C3] The pocket-sized therapeutic applicator according to paragraph [C1], wherein a single application of the anhydrous composition applied to the user's lips amounts to 25% or less than a daily recommended dose limit of the natural botanical nutritional supplement.

[C4] The pocket-sized therapeutic applicator according to paragraph [C1], wherein at least 5% of the natural botanical nutritional supplement included in a single application of the anhydrous composition applied to the user's lips is absorbed and/or ingested into the user's body.

[C5] The pocket-sized therapeutic applicator according to paragraph [C1], wherein the natural botanical essential oil scent portion includes at least one of: Chamomile, Lavender, Zesty Grapefruit, Mandarin, Cacao, Vanilla, Lemon, Peppermint, Lime, and Clary Sage.

[C6] The pocket-sized therapeutic applicator according to paragraph [C1], wherein the natural botanical nutritional supplement portion includes at least one of: Valerian Root, Ginseng, Maca, Ashwagandha and St. John's Wort.

[C7] The pocket-sized therapeutic applicator according to paragraph [C1], wherein the natural botanical essential oil scent portion and the natural botanical nutritional supplement portion include one of the combinations of: Chamomile, Lavender, and Valerian Root; Zesty Grapefruit, Mandarin and Ginseng; Cacao, Vanilla and Maca; Lemon, Peppermint and Ashwagandha; and Lime Clary Sage and St. John's Wort.

[C8] The pocket-sized therapeutic applicator according to paragraph [C1], wherein the applicator is one of the following: stick container actuated with a thumb wheel and threaded rod operatively associated with a base mechanism with a cap; squeezable tube container operatively associated with a pinhole dispenser mechanism with a cap; a vial container operatively associated with a roller ball dispenser mechanism with a cap; and a jar container including a removable top.

[C9] The pocket-sized therapeutic applicator according to paragraph [C1], wherein the wellness benefit includes at least one of : sleep, relaxation, stress-relief, anxiety relief, calm, energy, awakening, libido, sexual pleasure, and mood.

[C10] The pocket-sized therapeutic applicator according to paragraph [C1], wherein the natural botanical essential oil scent portion is a least 5× and no more than 15× of the natural botanical nutritional supplement portion; the natural botanical nutritional supplement included in a single application of the anhydrous composition to the user's lips amounts to 25% or less than a daily recommended dose limit; at least 5% of the natural botanical nutritional supplement included in a single application of the anhydrous composition to the user's lips is absorbed and/or ingested into the user's body, and a total natural botanical nutritional supplement content of the anhydrous composition included in an applicator is less than 100% of the daily recommended dose limit.

[D1] A method for a user to receive a self-administered oral dose of a nutritional supplement while simultaneously receiving nasal aromatherapy, the method comprising: the user applying one of a balm and an ointment of composition to the user's lips, the composition including a combination of a wax and oil base portion, a scent portion and a nutritional supplement portion, the wax and oil base portion amounting to at least 50% of a total weight of the composition, the scent portion amounting to 30% or less of the total weight of the composition, and the nutritional supplement portion amounting to 20% or less of the total weight of the composition, wherein, after applying the composition to the user's lips, at least part of the nutritional supplement portion is absorbed and/or ingested into the user's body for a wellness benefit distinct from any topical benefit related to a physical condition of the user's lips, and the scent portion simultaneously provides nasal aromatherapy to the user.

[E1] A composition applied to a user's lips to orally deliver a nutritional supplement and simultaneously nasally deliver aromatherapy, the composition comprising: one of a balm and an ointment of composition applied to the user's lips, the composition including a combination of a wax and oil base portion, a scent portion and a nutritional supplement portion, the wax and oil base portion amounting to at least 50% of a total weight of the composition, the scent portion amounting to 30% or less of the total weight of the composition, and the nutritional supplement portion amounting to 20% or less of the total weight of the composition, wherein, after applying the composition to the user's lips, at least part of the nutritional supplement portion is absorbed and/or ingested into the user's body for a wellness benefit distinct from any topical benefit related to a physical condition of the user's lips, and the scent portion simultaneously provides nasal aromatherapy to the user.

[F1] A pocket-sized therapeutic applicator to orally deliver nutritional supplements to a user while simultaneously providing aromatherapy to the user, the applicator comprising: one of a balm and an ointment of composition applied to the user's lips, the composition including a combination of a wax and oil base portion, a scent portion and a nutritional supplement portion, the wax and oil base portion amounting to at least 50% of a total weight of the composition, the scent portion amounting to 30% or less of the total weight of the composition, and the nutritional supplement portion amounting to 20% or less of the total weight of the composition; and an applicator housing containing the composition and dispensing the composition for application to the user's lips, wherein, after applying the composition to the user's lips, at least part of the nutritional supplement portion is absorbed and/or ingested into the user's body for a wellness benefit distinct from any topical benefit related to a physical condition of the user's lips, and the scent portion simultaneously provides nasal aromatherapy to the user.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A method for a user to receive a self-administered oral dose of a natural botanical nutritional supplement while simultaneously receiving nasal aromatherapy, the method comprising:
the user applying one of a balm and an ointment of anhydrous composition to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition,
wherein, after applying the anhydrous composition the user's lips, at least a part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

2. The method of claim 1, wherein the natural botanical essential oil scent portion is a least 5× and no more than 15× of the natural botanical nutritional supplement portion.

3. The method of claim 1, wherein a single application of the anhydrous composition applied to the user's lips amounts to 25% or less than a daily recommended dose limit of the natural botanical nutritional supplement.

4. The method of claim 1, wherein at least 5% of the natural botanical nutritional supplement included in a single application of the anhydrous composition applied to the user's lips is absorbed and/or ingested into the user's body.

5. The method of claim 1, wherein the natural botanical essential oil scent portion includes at least one of: Chamomile, Lavender, Zesty Grapefruit, Mandarin, Cacao, Vanilla, Lemon, Peppermint, Lime and Clary Sage.

6. The method of claim 1, wherein the natural botanical nutritional supplement portion includes at least one of: Valerian Root, Ginseng, Maca, Ashwagandha and St. John's Wort.

7. The method of claim 1, wherein the natural botanical essential oil scent portion and the natural botanical nutritional supplement portion include one of the combinations of: Chamomile, Lavender, and Valerian Root; Zesty Grapefruit, Mandarin and Ginseng; Cacao, Vanilla and Maca; Lemon, Peppermint and Ashwagandha; and Lime Clary Sage and St. John's Wort.

8. The method of claim 1, wherein the applicator is one of the following:
a stick container actuated with a thumb wheel and threaded rod operatively associated with a base mechanism with a cap;
a squeezable tube container operatively associated with a pinhole dispenser mechanism with a cap;
a vial container operatively associated with a roller ball dispenser mechanism with a cap; and
a jar container including a removable top.

9. The method of claim 1, wherein the wellness benefit includes at least one of: sleep, relaxation, stress-relief, anxiety relief, calm, energy, awakening, libido, sexual pleasure, and mood.

10. The method of claim 1, wherein the natural botanical essential oil scent portion is a least 5× and no more than 15× of the natural botanical nutritional supplement portion; the natural botanical nutritional supplement included in a single application of the anhydrous composition to the user's lips amounts to 25% or less than a daily recommended dose limit; at least 5% of the natural botanical nutritional supplement included in a single application of the anhydrous composition to the user's lips is absorbed and/or ingested into the user's body, and a total natural botanical nutritional supplement content of the anhydrous composition included in an applicator is less than 100% of the daily recommended dose limit.

11. An anhydrous composition applied to a user's lips to orally deliver a nutritional supplement and simultaneously nasally deliver aromatherapy, the anhydrous composition comprising:
one of a balm and an ointment of anhydrous composition applied to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition,
wherein, after applying the anhydrous composition the user's lips, at least part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

12. The anhydrous composition according to claim 11, wherein the natural botanical essential oil scent portion is a least 5× and no more than 15× the natural botanical nutritional supplement portion.

13. The anhydrous composition according to claim 11, wherein a single application of the anhydrous composition applied to the user's lips amounts to 25% or less than a daily recommended dose limit of the natural botanical nutritional supplement.

14. The anhydrous composition according to claim 11, wherein at least 5% of the natural botanical nutritional supplement included in a single application of the anhydrous composition applied to the user's lips is absorbed and/or ingested into the user's body.

15. The anhydrous composition according to claim 11, wherein the natural botanical essential oil scent portion includes at least one of: Chamomile, Lavender, Zesty Grapefruit, Mandarin, Cacao, Vanilla, Lemon, Peppermint, Lime and Clary Sage.

16. The anhydrous composition according to claim 11, wherein the natural botanical nutritional supplement portion includes at least one of: Valerian Root, Ginseng, Maca, Ashwagandha and St. John's Wort.

17. The anhydrous composition according to claim 11, wherein the natural botanical essential oil scent portion and the natural botanical nutritional supplement portion include one of the combinations of: Chamomile, Lavender, and Valerian Root; Zesty Grapefruit, Mandarin and Ginseng; Cacao, Vanilla and Maca; Lemon, Peppermint and Ashwagandha; and Lime Clary Sage and St. John's Wort.

18. The anhydrous composition according to claim 11, wherein the anhydrous composition is applied to the user's lips using one of the following:
a stick container actuated with a thumb wheel and threaded rod operatively associated with a base mechanism with a cap;
a squeezable tube container operatively associated with a pinhole dispenser mechanism with a cap;
a vial container operatively associated with a roller ball dispenser mechanism with a cap; and
a jar container including a removable top.

19. The anhydrous composition according to claim 11, wherein the wellness benefit includes at least one of: sleep, relaxation, stress-relief, anxiety relief, calm, energy, awakening, libido, sexual pleasure, and mood.

20. The anhydrous composition according to claim 11, wherein the natural botanical essential oil scent portion is a least 5× and no more than 15× of the natural botanical nutritional supplement portion; the natural botanical nutritional supplement included in a single application of the anhydrous composition to the user's lips amounts to 25% or less than a daily recommended dose limit; at least 5% of the natural botanical nutritional supplement included in a single application of the anhydrous composition to the user's lips is absorbed and/or ingested into the user's body, and a total natural botanical nutritional supplement content of the anhydrous composition included in an applicator is less than 100% of the daily recommended dose limit.

21. A pocket-sized therapeutic applicator to orally deliver botanical nutritional supplements to a user while simultaneously providing aromatherapy to the user, the applicator comprising:
one of a balm and an ointment of anhydrous composition applied to the user's lips, the anhydrous composition including a combination of a natural wax and natural oil base portion, a natural botanical essential oil scent portion and a natural botanical nutritional supplement portion, the natural wax and natural oil base portion amounting to at least 50% of a total weight of the anhydrous composition, the natural botanical essential oil scent portion amounting to 30% or less of the total weight of the anhydrous composition, and the natural botanical nutritional supplement portion amounting to 20% or less of the total weight of the anhydrous composition; and
an applicator housing containing the anhydrous composition and dispensing the anhydrous composition for application to the user's lips,
wherein, after applying the anhydrous composition to the user's lips, at least part of the natural botanical nutritional supplement portion is absorbed and/or ingested into the user's body for a wellness benefit distinct from any topical benefit related to a physical condition of the user's lips, and the natural botanical essential oil scent portion simultaneously provides nasal aromatherapy to the user.

22. The pocket-sized therapeutic applicator according to claim 21, wherein the natural botanical essential oil scent portion is a least 5× and no more than 15× of the natural botanical nutritional supplement portion.

23. The pocket-sized therapeutic applicator according to claim 21, wherein a single application of the anhydrous composition applied to the user's lips amounts to 25% or less than a daily recommended dose limit of the natural botanical nutritional supplement.

24. The pocket-sized therapeutic applicator according to claim 21, wherein at least 5% of the natural botanical nutritional supplement included in a single application of the anhydrous composition applied to the user's lips is absorbed and/or ingested into the user's body.

25. The pocket-sized therapeutic applicator according to claim 21, wherein the natural botanical essential oil scent portion includes at least one of: Chamomile, Lavender, Zesty Grapefruit, Mandarin, Cacao, Vanilla, Lemon, Peppermint, Lime and Clary Sage.

26. The pocket-sized therapeutic applicator according to claim 21, wherein the natural botanical nutritional supplement portion includes at least one of: Valerian Root, Ginseng, Maca, Ashwagandha and St. John's Wort.

27. The pocket-sized therapeutic applicator according to claim 21, wherein the natural botanical essential oil scent portion and the natural botanical nutritional supplement portion include one of the combinations of: Chamomile, Lavender, and Valerian Root; Zesty Grapefruit, Mandarin and Ginseng; Cacao, Vanilla and Maca; Lemon, Peppermint and Ashwagandha; and Lime Clary Sage and St. John's Wort.

28. The pocket-sized therapeutic applicator according to claim 21, wherein the applicator is one of the following:
stick container actuated with a thumb wheel and threaded rod operatively associated with a base mechanism with a cap;
squeezable tube container operatively associated with a pinhole dispenser mechanism with a cap;
a vial container operatively associated with a roller ball dispenser mechanism with a cap; and
a jar container including a removable top.

29. The pocket-sized therapeutic applicator according to claim 21, wherein the wellness benefit includes at least one of: sleep, relaxation, stress-relief, anxiety relief, calm, energy, awakening, libido, sexual pleasure, and mood.

30. The pocket-sized therapeutic applicator according to claim 21, wherein the natural botanical essential oil scent portion is a least 5× and no more than 15× of the natural botanical nutritional supplement portion; the natural botanical nutritional supplement included in a single application of the anhydrous composition to the user's lips amounts to 25% or less than a daily recommended dose limit; at least 5% of the natural botanical nutritional supplement included in a single application of the anhydrous composition to the user's lips is absorbed and/or ingested into the user's body, and a total natural botanical nutritional supplement content of the anhydrous composition included in an applicator is less than 100% of the daily recommended dose limit.
